(19) United States
(12) Patent Application Publication
(10) Pub. No.: US 2025/0262095 A1
(43) Pub. Date: Aug. 21, 2025

(54) EARPLUG HAVING BAMBOO ATTENUATOR

(71) Applicant: Imam Abdulrahman Bin Faisal University, Dammam (SA)

(72) Inventors: Duaa ALABDULHADI, Dammam (SA); Hala EL WAKEEL, Dammam (SA); Ruba AL KHALDI, Dammam (SA)

(73) Assignee: Imam Abdulrahman Bin Faisal University, Dammam (SA)

(21) Appl. No.: 18/581,197

(22) Filed: Feb. 19, 2024

(51) Int. Cl. A61F 11/08

(52) U.S. Cl. CPC A61F 11/085

(57) ABSTRACT

An earplug having a bamboo attenuator and a tapered body portion that includes a central shaft with bamboo attenuator disposed within and extending throughout an entirety of a hollow sheath, and contact structures which are flexible and have a successively smaller size moving from a lateral end of the tapered body portion to a medial end of the tapered body portion, and a stopper portion attached to the lateral end of the tapered body portion. The earplug provides passive, nonlinear attenuation of incoming sound to provide hearing protection to a user while allowing for comfortable use.

### BACKGROUND OF THE INVENTION

### Field of the Invention

The present disclosure relates to an earplug including a bamboo attenuator which may be useful as a hearing protection device.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

There are many situations in which persons are exposed to loud sounds at sound pressure levels sufficient to cause permanent hearing damage. To minimize the degree of hearing damage incurred in these situations, hearing protectors in the form of earplugs, earmuffs, or a combination of earplugs and earmuffs can be worn. However, in many situations where loud sounds are prevalent, verbal communication between persons is critical for factors such as safety. For example, in a work environment with heavy machinery, the large engines or activity of the heavy machinery can produce sound levels loud enough to damage hearing, particularly over long exposure times. Hearing protection is critical for the short- and long-term health of workers in that environment. Equally critical to the health of such workers is being able to communicate and coordinate with other workers. Such communication is also critical for efficient work.

Because of the possibility that a worker near heavy machinery will need to wear hearing protection for an extended period of time, such as a full 8-hour shift, user comfort is critical. Uncomfortable protective equipment can have the unintended effect of lower user compliance. Users may not properly use the protective equipment or may forego using it altogether. In the context of hearing protection, a user experiencing discomfort may move the hearing protection to a more comfortable position that results in less effective protection or may remove the hearing protection.

Accordingly, it is an objective of the present of the present disclosure to provide a hearing protecting earplug.

### SUMMARY OF THE INVENTION

The present disclosure relates to an earplug, comprising a tapered body portion that includes a central shaft comprising a hollow sheath and a bamboo attenuator disposed within the hollow sheath and a plurality of contact structures arranged about an exterior of the central shaft, the contact structures being flexible and having a successively smaller size moving from a lateral end of the tapered body portion to a medial end of the tapered body portion, as well as a stopper portion attached to the lateral end of the tapered body portion, wherein the earplug is configured to be placed in an ear such that the medial end of the tapered body portion is securely disposed within an ear canal by contact between the contact structures and an interior surface of the ear canal, the stopper portion is rigid and securely disposed in an exterior portion of the ear, and the bamboo attenuator is a rod which extends throughout an entirety of a length of the central shaft.

In some embodiments, the earplug comprises at least three contact structures.

In some embodiments, the contact structures are formed from an elastomer.

In some embodiments, the elastomer is a silicone elastomer.

In some embodiments, the stopper portion is formed from a rigid material.

In some embodiments, the rigid material is a rigid rubber.

In some embodiments, each of the contact structures is disposed about an entirety of a circumference of the central shaft.

In some embodiments, the contact structures have an elliptical shape about the exterior of the central shaft.

In some embodiments, the contact structures have a rounded trough cross-sectional shape having a rounded trough bottom oriented toward the medial end of the tapered body portion and an open trough top oriented toward the lateral end of the tapered body portion. In some embodiments, the bamboo attenuator is a cylindrical rod.

In some embodiments, the bamboo attenuator has a diameter of 1.5 to 5 mm.

In some embodiments, the earplug is devoid of electrical components.

In some embodiments, the tapered body portion has a length of 15.0 to 20.0 mm. In some embodiments, the earplug has a total length of 18.0 to 28.0 mm.

In some embodiments, a lateralmost contact structure has a major axis of 11.0 to 17.0 mm.

In some embodiments, a medialmost contact structure has a major axis of 5.0 to 10.0 mm.

In some embodiments, the earplug has a threshold attenuation level of 60 to 100 db.

The present application also relates to a hearing protection device, comprising a first earplug as described above, a second earplug as described above, and a connecting cord attached to the stopper portion of each of the first earplug and the second earplug.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows a perspective rendering of a rear three-quarters view of an earplug according to the present disclosure;

FIG. 2 shows a perspective rendering of front three-quarters view of an earplug according to the present disclosure;

FIG. 3 shows an alternate perspective rendering of a rear three-quarters view of an earplug according to the present disclosure;

FIG. 4 shows a schematic drawing of a front three-quarters view of an earplug according to the present disclosure;

FIG. 5 shows a schematic drawing of a side-view of an earplug according to the present disclosure;

FIG. 6 shows a front view of an earplug according to the present disclosure; and

FIG. 7 shows a top-down view of an earplug according to the present disclosure.

### DETAILED DESCRIPTION OF THE INVENTION

In the following description, it is understood that other embodiments may be utilized and structural and operational changes may be made without departure from the scope of the present embodiments disclosed herein.

### Definitions

As used herein the words “a” and “an” and the like carry the meaning of “one or more.”

As used herein, the terms “optional” or “optionally” means that the subsequently described event(s) can or cannot occur or the subsequently described component(s) may or may not be present (e.g., 0 wt. %).

According to a first aspect, the present disclosure relates to an earplug having a tapered body portion and a stopper portion. The tapered body portion comprises central shaft that includes a hollow sheath. This hollow sheath may take the form of a tube which defines an interior channel. The hollow sheath preferably extends throughout an entirety of the tapered body portion. That is, the hollow sheath defines an interior channel which extends from a medial end of the tapered body portion to a lateral end of the tapered body portion.

In general, the hollow sheath can have any suitable shape. For example, the hollow sheath can have an exterior shape which is substantially circular, elliptical, polygonal, or other shape. Similarly, the hollow sheath can have an interior shape which is substantially circular, elliptical, polygonal, or other shape. The hollow sheath can have an interior shape which is the same as the exterior shape or is different from the exterior shape. For example, the hollow sheath can take the form of a cylindrical tube having a circular exterior shape and a circular interior shape. In another example, the hollow sheath can take the form of a hexagonal tube having a hexagonal exterior shape and a circular interior shape. In some embodiments, the hollow sheath has a medial end which is open. That is, the hollow sheath has a tubular shape having an unobstructed medial end. In some embodiments, the hollow sheath has a lateral end which is open. In preferred embodiments, the hollow sheath is open on both a medial and a lateral end.

In some embodiments, the hollow sheath has a constant cross-sectional size. That is, the cross-sectional size does not vary along a length of the hollow sheath. In some embodiments where the hollow sheath is cylindrical, the cylindrical shape has a constant diameter. In some embodiments, the hollow sheath does not have a constant cross-sectional size. That is, the cross-sectional size does vary along a length of the hollow sheath. Such variation may be in form of a linear change (i.e., with a constant rate of change of cross-sectional size), a stepwise change, a curved change (i.e., with a non-constant rate of change of cross-sectional size), or a combination of these.

In general, the hollow sheath may be flexible or rigid. In some embodiments, the hollow sheath is rigid. A rigid hollow sheath may be constructed of any suitable rigid material. Examples of rigid materials which may be used in the construction of the hollow sheath include, but are not limited to, wood, bamboo, rigid polymers such as acrylonitrile butadiene styrene (ABS), polyethylene, polypropylene, polyacrylate, polymethacrylate, poly(methyl methyacrylate), polyacrylic acid, poly amide, polybutylene, polycarbonate, polyvinylchloride, and the like, metal and metal alloys, and rigid rubber such as styrene-butadiene rubber, isoprene rubber, nitrile rubber, and neoprene rubber. In some embodiments, the rigid materials comprises two or more rigid materials. In some embodiments, the rigid hollow sheath comprises an elastomeric coating. In general, the elastomeric coating can include or be formed from a suitable elastomeric material as described below.

In some embodiments, the tapered body portion has a length of 15.0 to 20.0 mm, preferably 15.5 to 19.5 mm, preferably 16.0 to 19.0 mm, preferably 16.25 to 18.5 mm, preferably 16.5 to 18.0 mm, preferably 16.75 to 17.5 mm, preferably 16.9 to 17.1 mm, preferably 17.0 mm.

The tapered body portion also comprises a bamboo attenuator disposed within the hollow sheath. The bamboo attenuator can have any suitable cross-sectional shape and be any suitable size such that it fits within the hollow sheath. Preferably, the bamboo attenuator has a cross-sectional shape which matches the interior shape of the hollow sheath. Such a match may be advantageous for securing the bamboo attenuator within the hollow sheath and/or for ensuring that sufficient contact is made between the bamboo attenuator and the hollow sheath to prevent sound transmission around the bamboo attenuator (e.g., through a gap between the hollow sheath and the bamboo attenuator). In some embodiments, the bamboo attenuator fills an entirety of a cross-sectional area of the hollow sheath. That is, there are no gaps between the bamboo attenuator and an interior surface of the hollow sheath. In some embodiments, the bamboo attenuator does not fill an entirety of a cross-sectional area of the hollow sheath.

The bamboo attenuator may have any overall shape. In some embodiments, the bamboo attenuator takes the form of a solid cylinder or prism. For example, the bamboo attenuator may be substantially rod-shaped with a circular cross-section or a polygonal cross-section. The solid cylinder or prism may be substantially free of interior channels, voids, or spaces. In some embodiments, the solid cylinder or prism is disposed throughout an entirety of the hollow sheath. In some embodiments, the bamboo attenuator takes the form of a solid cylinder or prism that is not disposed throughout the entirety of the hollow sheath. In some embodiments, two or more bamboo attenuators are disposed within the hollow sheath. For example, the hollow sheath may have two solid cylindrical bamboo attenuators disposed within. In this example, the two solid cylindrical bamboo attenuators may be disposed abutting each other. That is, there is not a gap between the two solid cylindrical bamboo attenuators. In this example, the two bamboo attenuators may together extend throughout an entirety of the hollow sheath. In another example, the two solid cylindrical bamboo attenuators may be disposed such that there is a gap between the two solid cylindrical bamboo attenuators. Such a gap may be useful for additional sound dampening. In some embodiments, the bamboo attenuator may be substantially disc-shaped. That is, the bamboo attenuator has a length that is smaller than a width of the bamboo attenuator. In some embodiments, multiple disc-shaped bamboo attenuators are disposed throughout the hollow sheath. Such disc-shaped bamboo attenuators may be disposed abutting one another (i.e., without a gap between them), with a gap between adjacent bamboo attenuators, or a combination of these.

In general the bamboo attenuator may be constructed of a bamboo-comprising material. In some embodiments, the bamboo-comprising material comprises greater than 50% bamboo by mass. In some embodiments, the bamboo-comprising material is bamboo wood. Such bamboo wood may be any suitable bamboo wood. The bamboo wood may be a single piece of bamboo wood or may be multiple pieces of bamboo wood. When the bamboo attenuator is constructed of or contains bamboo wood formed from multiple pieces, those multiple pieces may be joined by any suitable method or technique known to one of ordinary skill in the art. For example, the pieces of bamboo wood may be joined by gluing with an adhesive, joined by or encased in a polymer resin, or treated by a process involving compression or lamination to be joined without the use of non-bamboo components. In some embodiments, the bamboo-comprising material is a bamboo composite. For example, the bamboo composite may be a compressed and thermally treated bamboo strip composite, bamboo lumber, bamboo laminate, or bamboo fiber composite. In one example, the bamboo attenuator is formed from compressed bamboo fibers. In some embodiments, the bamboo attenuator is a single piece of bamboo wood.

Bamboo wood is typically described by an orientation relative to the bamboo plant from which the bamboo wood is obtained. Generally, bamboo wood is described by three main directions. Radial orientation refers to an orientation directed radially outward or inward relative to the tube shape of the bamboo plant. Longitudinal orientation refers to an orientation directed along a length of the tube shape of the bamboo plant. Tangential orientation refers to an orientation directed along a vector tangent to the tube shape of the bamboo plant. In general, the bamboo wood may be of radial orientation, tangential orientation, longitudinal orientation, or a mixture of these. In some embodiments, the bamboo attenuator is described by a bamboo wood orientation along the length of the hollow sheath. In some embodiments, the bamboo attenuator has a longitudinal orientation along the length of the hollow sheath. The longitudinal orientation of bamboo wood is typically associated with pores or channels formed by various tissues of the bamboo plant, such as vascular bundles and fiber sheaths. When the longitudinal orientation of bamboo wood is directed along a length of the hollow sheath, the pores or channels formed by various tissues of the bamboo plant are also oriented along the length of the hollow sheath. This orientation match may be advantageous for creating small pores or channels that run through some or all of the bamboo attenuator as described below. These pores or channels may be advantageous for low-intensity sound transmission or transduction, air exchange, moisture exchange, or a combination of these.

In general, the bamboo may be any species of bamboo. Examples of species of bamboo include, but are not limited to Acidosasa chienouensis, Acidosasa edulis, Actinocladum verticillatum, Ampelocalamus scandens, Apoclada simplex, Arthrostylidium angustifolium, Arthrostylidium auriculatum, Arthrostylidium banaoense, Arthrostylidium canaliculatum, Arthrostylidium chiribiquetense, Arthrostylidium cubense, Arthrostylidium distichum, Arthrostylidium ecuadorense, Arthrostylidium ekmanii, Arthrostylidium excelsum, Arthrostylidium farctum, Arthrostylidium fimbriatum, Arthrostylidium fimbrinodum, Arthrostylidium grandifolium, Arthrostylidium haitiense, Arthrostylidium judziewiczii, Arthrostylidium longiflorum, Arthrostylidium merostachyoides, Arthrostylidium multispicatum, Arthrostylidium obtusatum, Arthrostylidium pubescens, Arthrostylidium punctulatum, Arthrostylidium reflexum, Arthrostylidium sarmentosum, Arthrostylidium scandens, Arthrostylidium schomburgkii, Arthrostylidium simpliciusculum, Arthrostylidium urbanii, Arthrostylidium venezuelae, Arthrostylidium virolinense, Arthrostylidium youngianum, Arundinaria appalachiana, Arundinaria funghomii, Arundinaria gigantea, Arundinaria gigantea Macon, Arundinaria tecta, Athroostachys capitata, Aulonemia queko, Bambusa arnhemica, Bambusa balcooa, Bambusa bambos, Bambusa basihirsuta, Bambusa beecheyana, Bambusa beecheyana var. pubescens, Bambusa blumeana, Bambusa boniopsis, Bambusa burmanica, Bambusa chungii, Bambusa chungii var. barbelatta, Bambusa cornigera, Bambusa dissimulator, Bambusa dissimulator Albinodia, Bambusa distegia, Bambusa dolichoclada, Bambusa dolichoclada Stripe, Bambusa dolichomerithalla, Bambusa emeiensis Chrysotrichus, Bambusa emeiensis Flavidovirens, Bambusa emeiensis Viridiflavus, Bambusa eutuldoides, Bambusa eutuldoides Viridivittata, Bambusa flexuosa, Bambusa fulda, Bambusa gibba, Bambusa glaucophylla, Bambusa intermedia, Bambusa lako, Bambusa lapidea, Bambusa longispiculata, Bambusa luteostriata, Bambusa maculata, Bambusa malingensis, Bambusa membranacea, Bambusa multiplex, Bambusa multiplex Alphonse Karr, Bambusa multiplex Fernleaf, Bambusa multiplex Fernleaf Stripestem, Bambusa multiplex Golden Goddess, Bambusa multiplex Goldstripe, Bambusa multiplex Midori Green, Bambusa multiplex Riviereorum, Bambusa multiplex Silverstripe, Bambusa multiplex Tiny Fern, Bambusa multiplex Tiny Fern Striped, Bambusa multiplex Willowy, Bambusa multiplex, Bambusa mutabilis, Bambusa nutans, Bambusa odashimae, Bambusa oldhamii, Bambusa oliveriana, Bambusa pachinensis, Bambusa pallida, Bambusa pervariabili, Bambusa pervariabilis Viridistriatus, Bambusa polymorpha, Bambusa rigida, Bambusa rutila, Bambusa sinospinosa, Bambusa sinospinosa Hirose, Bambusa sinospinosa Clone X, Bambusa sinospinosa Nana, Bambusa sinospinosa Polymorpha, Bambusa sinospinosa Richard Waldron, Bambusa stenostachya, Bambusa textilis, Bambusa textilis Dwarf, Bambusa textilis Kanapaha, Bambusa textilis Maculata, Bambusa textilis Mutabilis, Bambusa textilis Scranton, Bambusa textilis var. albostriata, Bambusa textilis var. glabra, Bambusa textilis var. gracilis, Bambusa tulda, Bambusa tulda Striata, Bambusa tuldoides, Bambusa variostriata, Bambusa ventricosa, Bambusa Ilentricose Golden Buddha, Bambusa ventricosa Kimmei, Bambusa ventricosa, Bambusa vulgaris, Bambusa vulgaris Vittata, Bambusa vulgaris Wamin, Bambusa vulgaris Wamin Striata, Bashania fargesii, Bashania qingchengshanensis, Borinda albocerea, Borinda angustissima, Borinda contracta, Borinda frigidorum, Borinda fungosa, Borinda fungosa White cloud, Borinda grossa, Borinda KR 5288, Borinda lushuiensis, Borinda macclureana, Borinda nujiangensis, Borinda papyrifera, Borinda perlonga, Borinda sinospinosa Muliensis (sinospinosa often abbreviated sp.), Borinda yulongshanensis, Cathariostachys capitata, Cathariostachys madagascariensis, Cephalostachyum pergracile, Cephalostachyum viguieri, Cephalostachyum virgatum, Chimonobambusa macrophylla, Chimonobambusa macrophylla var. macrophylla, Chimonobambusa macrophylla var. leiboensis, Chimonobambusa marmorea, Chimonobambusa marmorea Variegata, Chimonobambusa marmorea, Chimonobambusa pachystachys, Chimonobambusa quadrangularis, Chimonobambusa quadrangularis Joseph de Jussieu, Chimonobambusa quadrangularis Suow, Chimonobambusa quadrangularis Yellow Groove, Chimonobambusa quadrangularis, Chimonobambusa szechuanensis, Chimonobambusa tumidissinoda, Chimonobambusa yunnanensis, Chimonocalamus pallens, Chusquea asymmetrica, Chusquea andina, Chusquea andina Blue Andes, Chusquea circinata, Chusquea circinata Chiapas, Chusquea coronalis, Chusquea culeou, Chusquea culeou Argentina, Chusquea culeou Caña Prieta, Chusquea culeou Hillier's Form, Chusquea culeou, Chusquea cumingii, Chusquea delicatula, Chusquea elata, Chusquea elegans, Chusquea falcata, Chusquea foliosa, Chusquea galeottiana, Chusquea gigantea, Chusquea glomerata, Chusquea glauca, Chusquea liebmannii, Chusquea leonardiorum, Chusquea loxensis, Chusquea maclurei, Chusquea macrostachya, Chusquea mimosa subsp. Australis, Chusquea montana, Chusquea muelleri, Chusquea nana, Chusquea pittieri, Chusquea simpliciflora, Chusquea simpliciflora Chiconquiaco, Chusquea simpliciflora Las Vigas, Chusquea subtilis, Chusquea sulcata, Chusquea tomentosa, Chusquea uliginosa, Chusquea valdiviensis, Chusquea virgata Chusquea sp., Davidsea attenuata, Decaryochloa diadelpha, Dendrocalamus asper, Dendrocalamus asper Betung Hitam, Dendrocalamus brandisii, Dendrocalamus brandisii Black, Dendrocalamus brandisii variegated, Dendrocalamus calostachyus, Dendrocalamus giganteus, Dendrocalamus giganteus Quail Clone, Dendrocalamus giganteus variegated, Dendrocalamus hamiltonii, Dendrocalamus jianshuiensis, Dendrocalamus jianshuiensis variegated, Dendrocalamus latiflorus, Dendrocalamus latiflorus Mei-nung, Dendrocalamus latiflorus Subconvex, Dendrocalamus longispathus, Dendrocalamus maroochy, Dendrocalamus minor, Dendrocalamus minor var. amoenus, Dendrocalamus minor var. minor, Dendrocalamus sikkimensis, Dendrocalamus sinicus, Dendrocalamus sp. Parker's Giant, Dendrocalamus strictus, Dendrocalamus Validus, Dendrocalamus xishuangbannaensis, Dendrocalamus yunnanicus, Dinochloa malayana, Dinochloa scandens, Dracaena sandriana (lucky bamboo), Drepanostachyum falcatum, Drepanostachyum falcatum var. sengteeanum, Drepanostachyum khasianum, Drepanostachyum microphyllum, Drepanostachyum sengteeanum, Eremitis monothalamia, Eremitis parviflora, Fargesia adpressa, Fargesia apircirubens, Fargesia apircirubens White Dragon, Fargesia communis, Fargesia denudata, Fargesia denudata Xian 1, Fargesia dracocephala, Fargesia murielae, Fargesia murielae Bimbo, Fargesia murieliae Harewood, Fargesia murieliae Jonny's Giant, Fargesia murieliae Jumbo Jet, Fargesia murieliae Simba, Fargesia murieliae SABE 939, Fargesia murieliae Vampire, Fargesia murieliae, Fargesia nitida, Fargesia robusta, Fargesia rufa, Ferrocalamus strictus, Filgueirasia Arenicola, Filgueirasia cannavieira, Gaoligongshania megalothyrsa, Gelidocalamus fangianus, Gigantochloa achmadii, Gigantochloa albociliata, Gigantochloa albopilosa, Gigantochloa albovestita, Gigantochloa andamanica, Gigantochloa apus, Gigantochloa aspera, Gigantochloa atter, Gigantochloa atroviolacea, Gigantochloa auriculata, Gigantochloa aya, Gigantochloa baliana, Gigantochloa balui, Gigantochloa calcicola, Gigantochloa cochinchinensis, Gigantochloa compressa, Gigantochloa densa, Gigantochloa dinhensis, Gigantochloa felix, Gigantochloa hasskarliana, Gigantochloa hayatae, Gigantochloa heteroclada, Gigantochloa heterostachya, Gigantochloa hirtinoda, Gigantochloa holttumiana, Gigantochloa hosseusii, Gigantochloa kachinensis, Gigantochloa kathaensis, Gigantochloa kuring, Gigantochloa kurzii, Gigantochloa latifolia, Gigantochloa latispiculata, Gigantochloa levis, Gigantochloa ligulate, Gigantochloa longiprophylla, Gigantochloa luteostriata, Gigantochloa macrostachya, Gigantochloa magentea, Gigantochloa maxima, Gigantochloa membranoidea, Gigantochloa merrilliana, Gigantochloa mogaungensis, Gigantochloa multiculmis, Gigantochloa nigrociliata, Gigantochloa novoguineensis, Gigantochloa papyracea, Gigantochloa parviflora, Gigantochloa parvifolia, Gigantochloa poilanei, Gigantochloa pruriens, Gigantochloa pseudoarundinacea, Gigantochloa pubinervis, Gigantochloa pubipetiolata, Gigantochloa ridleyi, Gigantochloa robusta, Gigantochloa rostrata, Gigantochloa scortechinii, Gigantochloa scribneriana, Gigantochloa serik, Gigantochloa sinuata, Gigantochloa stocksii, Gigantochloa tekserah, Gigantochloa tenuispiculata, Gigantochloa thoi, Gigantochloa tomentosa, Gigantochloa toungooensis, Gigantochloa velutina, Gigantochloa verticillate, Gigantochloa vietnamica, Gigantochloa vinhphuica, Gigantochloa wallichiana, Gigantochloa wanet, Gigantochloa wrayi, Gigantochloa wunthoensis, Gigantochloa yunzalinensis, Glaziophyton mirabile, Guadua paniculata, Hickelia madagascariensis, Himalayacalamus asper, Himalayacalamus cupreus, Himalayacalamus falconeri, Himalayacalamus falconeri, Himalayacalamus hookerianus, Himalayacalamus hookerianus, Himalayacalamus porcatus, Hibanobambusa tranquillans, Hitchcockella baronii, Indocalamus latifolius, Indocalamus longiauritus, Indocalamus tessellatus, Indosasa crassifolia, Leptocanna chinensis, Melocanna baccifera, Ochlandra strulata, Otatea acuminata, Phyllostachys acuta, Phyllostachys angusta, Phyllostachys arcana, Phyllostachys atrovaginata, Phyllostachys aurea, Phyllostachys aureosulcata, Phyllostachys bambusoides. Phyllostachys bissetii, Phyllostachys decora, Phyllostachys densiflorum, Phyllostachys dulcis, Phyllostachys edulis, Phyllostachys flexuosa, Phyllostachys glauca, Phyllostachys heteroclada, Phyllostachys humilis, Phyllostachys iridescens, Phyllostachys meyeri, Phyllostachys nidularia, Phyllostachys nigra, Phyllostachys nuda, Phyllostachys parvifolia, Phyllostachys rubromarginata, Phyllostachys violascens, Phyllostachys virella, Phyllostachys viridiglaucescens, Phyllostachys viridis, Phyllostachys viridis, Phyllostachys vivax, Pleioblastus argenteostriatus, Pleioblastus chino, Pleioblastus distichus, Pleioblastus fortune, Pleioblastus gramineus, Pleioblastus humilis, Pleioblastus hindsii, Pleioblastus humilis, Pleioblastus linearis, Pleioblastus simonii, Pleioblastus virens, Pleioblastus viridistriatus, Pseudosasa amabilis, Pseudosasa japonica, Pseudosasa japonica var. tsutsumiana, Pseudosasa usawai, Pseudostachyum polymorphum, Rhipidocladum harmonicum, Sasa borealis, Sasa kurilensis, Sasa nagimontana, Sasa oshidensis, Sasa palmata, Sasa tsuboiana, Sasa veitchii, Sasa veitchii, Sasaella albostriata, Sasaella glabra, Sasaella masamuneana, Sasaella ramose, Schizostachyum brachycladum, Schizostachyum chinense, Schizostachyum glaucifolium, Schizostachyum lima, Semiarundinaria densiflora, Semiarundinaria densiflora var. villosum, Semiarundinaria fastuosa, Semiarundinaria fortis, Semiarundinaria makinoi, Semiarundinaria okuboi, Semiarundinaria yamadori, Semiarundinaria yashadake, Semiarundinaria Yoshi, Shibataea kumasaca, Shibataea lancifolia, Sinobambusa tootsik, Thamnocalamus aristatus, Thamnocalamus tessellatus, Thyrsostachys siamensis, Valiha diffusa, Valiha perrieri, Yushania alpina, Yushania anceps, Yushania chungii, Yushania maling, and Yushania megalothyrsa.

The bamboo can be any part or combination of parts of a bamboo plant For example, the bamboo can be made using whole plants, roots, stems, leaves, flowers, bark, bulbs, fruits, seeds, buds, culum, node, internode, or any combination thereof. In bamboo, the culm refers to the part between the roots and branches, which makes up approximately two thirds of the plant. In general, three elements can be differentiated in the inner part of the culm: fibers, ducts or metaxylem vessels, and parenchyma. The fibers are the structural part of the plant, providing the required strength to stand upright and cope with the external mechanical stress. The ducts ensure the circulation of water and nutrients, while the parenchyma cells participate in food storage. Additionally, lignin keeps this network of cells, conduits, and fibers stuck together. The distribution of these elements in terms of percentages and densities depends not only on the species analyzed, but also on the part of the culm section that is being analyzed.

Typically, vascular bundles constitute a pattern that is repeated throughout the transversal section of the bamboo culm. The form and distribution of the fibers, metaxylem vessels, and ducts of the culm is typically maintained from the base of the culum to the end. However, this pattern may be slightly affected near the nodes, where more radial contact appears between the fibers and the ducts, thus giving the plant a greater resistance to torsional stresses. In the internodes, both the ducts and the fibers are predominantly axially (longitudinally) oriented, while the nodes provide the main transverse interconnections, though there are openings in the sides of the metaxylem vessels and ducts which permit some transmission of fluids and gasses to surrounding cells and adjacent vessels and ducts. Conducting tissue including metaxylem vessels and ducts has the largest conduit pores in the bamboo with a size of ˜100 μm in diameter and 0.5-0.9 mm in length. No obvious partition appears in the longitudinal direction of the conducting tissue. The parenchyma cells and vascular bundles are connected with many pits, which are distributed in their internal walls with a size ranging from sub-microns to a few microns. Parenchyma cells have a relatively closed lumen with a medium size of 14 to 40 μm in diameter and 0.8 to 1.6 μm in length, whereas fibers have the smallest pores with a size of ˜100 nm in diameter and 1.6 to 3.1 mm in length. The abundant pits in the bamboo scaffold would facilitate sufficient interior diffusion into the cell walls. By contrast, little tissue with straight conduits and high interconnectivity can be found in the radial and tangential directions. Thus, the transmission of gas or fluids into the interior of bamboo from the surface in the radial and tangential directions is primarily accomplished through pits. Longitudinally-oriented bamboo wood is typically associated with increased porosity compared to radially or transverse oriented bamboo as the ducts are oriented to provide channels or pores that are correctly oriented to run throughout the bamboo wood.

Bamboo wood typically has the following properties: a Janka hardness of 1,410 to 1,610 lbf (6,270 to 7,170 N), a rupture modulus of 11,020 to 24,450 lbf/in² (76 to 168.6 GPa), an elastic modulus: 2,610,000 to 2,900,000 lbf/in² (18 to 20 GPa), and a crushing strength of 8,990 to 13,490 lbf/in² (62 to 93 MPa).

Cellulose, hemicelluloses and lignin are the three major chemical compositions of bamboo. They contribute about 85 to 95% of the total bamboo mass. The minor components include pigments, tannins, protein, fat, pectin, ash, resins, waxes and inorganic salts. The content of cellulose in bamboo typically ranges from about 40% to 60%, not being much different from traditional wood, with 40% to 52% in conifers and 38% to 56% in angiosperms. Bamboo species typically have a holocellulose content (cellulose+hemicellulose) ranging from 60% to 70% and a lignin from 20% to 30%. However, the exact composition may change according to the species, growth conditions, age and part of the culm.

In some embodiments, the bamboo attenuator is disposed such that a medialmost surface of the bamboo attenuator (e.g., a top or bottom of a solid cylindrical attenuator) is disposed at a medial opening of the hollow sheath. In some embodiments, the bamboo attenuator is disposed such that the medialmost surface is disposed a distance within the hollow sheath. That is, there is a small distance between the medial opening of the hollow sheath and the bamboo attenuator. This may be referred to as a “recessed attenuator” or “medially recessed attenuator”. A small medial recess of the bamboo attenuator may be advantageous for ensuring that the bamboo attenuator remains clean, is capable of being easily cleaned, is not damaged, and/or does not create discomfort by contacting a user's ear canal.

In some embodiments, the bamboo attenuator is disposed such that a lateralmost surface of the bamboo attenuator (e.g., a top or bottom of a solid cylindrical attenuator) is disposed at a lateral opening of the hollow sheath. In some embodiments, the bamboo attenuator is disposed such that the lateralmost surface is disposed a distance within the hollow sheath. That is, there is a small distance between the medial opening of the hollow sheath and the bamboo attenuator. This may be referred to as a “recessed attenuator” or “laterally recessed attenuator”. A small lateral recess of the bamboo attenuator may be advantageous for ensuring that the bamboo attenuator remains clean, is capable of being easily cleaned, is not damaged, and/or does not accidentally come into contact with external objects.

In some embodiments, the bamboo attenuator has a diameter of 1.5 to 5.0 mm, preferably 1.75 to 4.5 mm, preferably 2.0 to 4.0 mm, preferably 2.25 to 3.75 mm, preferably 2.5 to 3.5 mm, preferably 2.75 to 3.25 mm, preferably 2.9 to 3.1 mm, preferably 3.0 mm. It should be noted that for bamboo attenuators having other cross-sectional shapes, a cross-sectional size in this range can be appropriate when adjusted to be for a similar measurement based on the cross-sectional shape. For example, a bamboo attenuator having an elliptical shape may have a major axis, minor axis, or average of major axis and minor axis in the stated size range. A bamboo attenuator having a square cross-sectional shape can have a side length or a diagonal in the stated size range.

The use of the bamboo attenuator may be associated with or cause distinct advantages. For example, the bamboo attenuator may be advantageous for allowing air and/or moisture exchange between the atmosphere and the ear canal while wearing the earplug. The bamboo attenuator may be advantageous for allowing moisture to be removed from the ear canal (but not exchanged with the atmosphere) while wearing the earplug. The bamboo attenuator may be advantageous for allowing pressure equalization between the ear canal and the atmosphere while wearing the ear plug. Mitigating excess moisture and/or pressure in the ear canal may improve the comfort of a user, particularly if the user is wearing the earplug for an extended period of time. The bamboo attenuator may be advantageous for providing a high level of sound attenuation, particularly at high sound intensities (high decibel levels or high transient pressure levels). This efficient attenuation may be particularly advantageous for forming an earplug with a high level of hearing protection for a user. The bamboo attenuator may be advantageous for simultaneously achieving a high level of hearing protection for a user while mitigating excess moisture and pressure in the ear canal as described above.

The tapered body portion also includes a plurality of contact structures arranged about an exterior of the central shaft. In some embodiments, the ear plug includes at least two contact structures. In some embodiments, the ear plug includes at least three contact structures. In some embodiments, the ear plug includes exactly three contact structures. The contact structures are configured to contact an interior surface of the ear canal. The contact structures may be useful for securing the earplug in the ear of a user.

In general, the contact structures may be formed of any suitable body-safe material. A body-safe material is any material which is generally recognized as safe for contact with the skin, preferably the skin of the ear and ear canal. A body-safe material may be hypoallergenic. Examples of materials suitable for forming the contact structures include, but are not limited to foams, rubbers, polymers, elastomers, and the like. Specific examples of suitable materials include, but are not limited to polyisoprene, polybutadiene, chloroprene, neoprene, butyl rubber, styrene-butadiene rubber, nitrile rubber, ethylene polypropylene rubber, epichlorohydrin rubber, acrylic rubber, silicone rubber, fluorosilicone rubber, fluoroelastomers such as FKM and FEPM, perfluoroelastomers such as FFKM, polyether block amides, ethylene-vinyl acetate, silicone elastomers such as polydimethylsiloxane (PDMS) or other linear polysiloxanes and network polysiloxanes such as those present in silicone resins, EPDM rubber, polyvinylchloride foam, polyurethane foam, latex foam, and combinations of these. The polyurethane foam may be a memory foam, also referred to as a “viscoelastic” polyurethane foam, or low-resilience polyurethane foam (LRPu). In a memory foam, the foam bubbles or ‘cells’ are typically open, effectively creating a matrix through which air can move. In some embodiments, the contact structures are substantially free of latex. In some embodiments, the earplug is substantially free of latex.

In preferred embodiments, the contact structures are flexible. In some embodiments, a contact structure being flexible refers to an entirety of the contact structure being flexible. In some embodiments, a contact structure being flexible refers to the contact structure having one or more portions that are flexible. Other portions of the contact structure may be inflexible. In general, different portions of a contact structure may be flexible with different measures of flexibility, such as a Young's modulus, flexural stiffness, spring constant, or other such measure. In some embodiments, a contact structure can have a progressive or gradient flexibility. The contact structures being flexible may be advantageous for allowing the contact structures to conform to the ear canal when being worn by a user. Such conforming may be advantageous for increasing the contact between the contact structures and ear canal, thereby increasing the security of the ear plug within the ear, or for increasing user comfort. Being flexible may be advantageous for allowing the contact structures to conform to the ear canal when being worn by a user. Such conforming may be advantageous for increasing the contact between the contact structures and ear canal, thereby increasing the security of the ear plug within the ear. Such conforming may be advantageous for increasing the comfort of the user. For example, the contact structures may flex to avoid placing pressure on the ear canal or contacting the ear canal with sharp edges, angles, or corners. In some embodiments, the contact structures have a Shore 00 hardness rating of 20 to 100, preferably 35 to 90, preferably 40 to 80. There are different Shore Hardness scales for measuring the hardness of different materials. The Shore 00 Hardness Scale measures rubbers and gels that are very soft. The Shore A Hardness Scale measures the hardness of flexible mold rubbers that range in hardness from very soft and flexible, to medium and somewhat flexible, to hard with almost no flexibility at all. Semi-rigid plastics can also be measured on the high end of the Shore A Scale. The Shore D Hardness Scale measures the hardness of hard rubbers, semi-rigid plastics and hard plastics.

In some embodiments, a contact structure is disposed about an entirety of a circumference of the central shaft. That is, there are no sections of an exterior of the central shaft about which the contact structure is not disposed in the region of that contact structure. In such embodiment, the contact structure preferably forms a continuous surface which contacts an entirety of the portion of the ear canal where the contact structure is situated. That is, there are no gaps for sound to pass between a portion of the contact structure and the ear canal. In some embodiments, the contact structures have an elliptical shape about the exterior of the central shaft. That is, when viewing the earplug end-on, the contact structure forms an ellipse about the central shaft. It should be noted here that “elliptical shape” also includes circular shapes. In the context of an elliptical shape, the contact structure should form a continuous ellipse, i.e., one that is free of gaps or discontinuities.

In some embodiments, the contact structures have a rounded trough cross-sectional shape having a rounded trough bottom oriented toward the medial end of the tapered body portion and an open trough top oriented toward the lateral end of the tapered body portion. This shape may also be described as a “flange”, “flange-shaped”, or other similar term. In some embodiments, the flange is oriented such that a free edge of the flange is oriented to point away from the medial end of the tapered body portion (i.e., the end that is situated and oriented in the ear canal of a user). In some embodiments, the flange shape is formed by a flat plane of flexible material oriented substantially perpendicular to the central shaft that bends or flexes away from the medial end of the earplug when the earplug is placed in the ear canal of a user.

In some embodiments, the earplug includes at least two contact structures. In some embodiments, the earplug includes at least three contact structures. In some embodiments, the earplug includes exactly three contact structures. Preferably, the contact structures have a successively smaller size moving from a lateral end of the tapered body portion to a medial end of the tapered body portion. Such a size is preferably measured as a distance from a center of the central shaft to a point of the contact structure farthest from the center of the central shaft. In the case of a contact structure having an elliptical shape, the size of the contact structure can be measured by the major axis of the ellipse. In some embodiments, the contact structures have a major axis of from 5.0 to 17.0 mm, preferably 5.5 to 16 mm, preferably 6.0 to 15.5 mm, preferably 6.5 to 15.0 mm, preferably 7.0 to 14.5 mm, preferably 7.5 to 14.0 mm. In some embodiments, a lateralmost contact structure (i.e. the contact structure closes to the lateral end of the tapered body portion) has a major axis of 11.0 to 17.0 mm, preferably 11.25 to 16.75 mm, preferably 11.5 to 16.5 mm, preferably 11.75 to 16.25 mm, preferably 12.0 to 16.0 mm, preferably 12.25 to 15.75 mm, preferably 12.5 to 15.5 mm, preferably 12.75 to 15.25 mm, preferably 13.0 to 15.0 mm, preferably 13.25 to 14.75 mm, preferably 13.5 to 14.5 mm, preferably 13.75 to 14.25 mm, preferably 13.9 to 14.1 mm, preferably 14.0 mm. It should be noted that based on the description of successively smaller sizes, the lateralmost contact structure should be the largest contact structure in terms of size as measured as a distance from a center of the central shaft to a point of the contact structure farthest from the center of the central shaft. In some embodiments, a medialmost contact structure (i.e. the contact structure closes to the medial end of the tapered body portion) has a major axis of 5.0 to 10.0 mm, preferably 5.25 to 9.75 mm, preferably 5.5 to 9.5 mm, preferably 5.75 to 9.25 mm, preferably 6.0 to 9.0 mm, preferably 6.25 to 8.75 mm, preferably 6.5 to 8.5 mm, preferably 6.75 to 8.25 mm, preferably 7.0 to 8.0 mm, preferably 7.25 to 7.75 mm, preferably 7.4 to 7.6 mm, preferably 7.5 mm. It should be noted that for contact structures having other shapes, a size in this range can be appropriate when adjusted to be for a similar measurement based on the shape. For example, a contact structure having a circular shape may have a diameter in the stated size range. In some embodiments, the successively smaller size of the contact structures defines the tapered shape of the tapered body portion. In some embodiments, there are gaps disposed between adjacent contact structures. Such gaps may be advantageous for preventing inadvertent overlapping or pinching of adjacent contact structures which may cause user discomfort or decreased sound attenuation.

The earplug also includes a stopper portion attached to the lateral end of the tapered body portion. In general, the stopper portion can be any suitable size and shape such that it does not enter the ear canal. Preferably, the stopper portion is of a suitable size and shape such that it prevents the tapered body portion from entering the ear canal to a depth to cause discomfort, pain, or damage to any part of the ear canal or ear drum of a user.

In some embodiments, the stopper portion is formed of a rigid material. Examples of rigid materials which may be used in the construction of the stopper portion include, but are not limited to, wood, bamboo, rigid polymers such as acrylonitrile butadiene styrene (ABS), polyethylene, polypropylene, polyacrylate, polymethacrylate, poly(methyl methyacrylate), polyacrylic acid, poly amide, polybutylene, polycarbonate, polyvinylchloride, and the like, metal and metal alloys, and rigid rubber such as styrene-butadiene rubber, isoprene rubber, nitrile rubber, and neoprene rubber. In some embodiments, the rigid material comprises two or more rigid materials. In some embodiments, stopper portion comprises an elastomeric coating. In general, the elastomeric coating can include or be formed from a suitable elastomeric material as described above. In some embodiments, the stopper portion is formed from a rigid rubber.

The tapered body portion is configured to be placed within the ear canal while the stopper portion remains outside of the ear canal. The medial end of the tapered body portion is configured to be placed into the ear canal and oriented in a medial direction of a person wearing the earplug (i.e., into the ear canal, toward the ear drum). The tapered body portion can also have a lateral end which is configured to be connected to or integrated with the stopper portion. When worn by a user, the lateral end of the tapered body portion can be located in the ear canal, at an entrance to the ear canal, or in an external portion of the ear. Similarly, when worn by a user, the stopper portion is configured to be located in an external portion of the ear. That is, the stopper portion does not enter the ear canal. Preferably, the stopper portion is securely disposed in an exterior portion of the ear.

In some embodiments, the earplug has a total length of 18.0 to 28.0 mm, preferably 18.5 to 27.5 mm, preferably 19.0 to 27.0 mm, preferably 19.5 to 26.5 mm, preferably 20.0 to 26.0 mm, preferably 20.5 to 25.5 mm, preferably 21.0 to 25.0 mm, preferably 21.5 to 24.5 mm, preferably 22.0 to 24.0 mm, preferably 22.25 to 23.75 mm, preferably 22.5 to 23.5 mm, preferably 22.75 to 23.25 mm, preferably 22.9 to 23.1 mm, preferably 23.0 mm.

In some embodiments, the attenuation characteristics of this type of earplugs are level-dependent. Level-dependent attenuation is also referred to as “nonlinear attenuation”. In level-dependent attenuation, only incoming sound waves at levels greater than some intensity level, for example 110 dB SPL, are attenuated. This intensity level at which attenuation begins may be referred to as a “threshold attenuation level” or some similar term. These nonlinear earplugs therefore provide protection against intense impulse noises such as gunfire, engine backfires, large impacts, and other noises which produces peak sound pressure levels in excess of the threshold attenuation level (e.g., 110 dB SPL), but transmit sounds unattenuated at levels below that threshold attenuation level. In some embodiments, the earplug has a threshold attenuation level of 60 to 100 db, preferably 62.5 to 97.5 db, preferably 65 to 95 db, preferably 67.5 to 92.5 db, preferably 70 to 90 db, preferably 72.5 to 87.5 db, preferably 75 to 85 db, preferably 77.5 to 82.5 db, preferably 79 to 81 db, preferably 80 db. In some embodiments, the earplug has a threshold attenuation level which is dependent on a diameter or other similar cross-sectional size measurement of the bamboo attenuator. In some embodiments, the bamboo attenuator has a diameter of 2.0 mm and the earplug has a threshold attenuation level of 60 to 76 db, preferably 62 to 74 db, preferably 64 to 72 db, preferably 66 to 70 db, preferably 67 to 69 db, preferably 68 db. In some embodiments, the bamboo attenuator has a diameter of 3.0 mm and the earplug has a threshold attenuation level of 70 to 80 db, preferably 71 to 79 db, preferably 72 to 78 db, preferably 73 to 77 db, preferably 74 to 76 db, preferably 75 db. In some embodiments, the bamboo attenuator has a diameter of 4.0 mm and the earplug has a threshold attenuation level of 78 to 100 db, preferably 80 to 98 db, preferably 82 to 96 db, preferably 84 to 94 db, preferably 86 to 92 db, preferably 88 to 90 db, preferably 89 db.

In some embodiments, the earplug does not have active noise reduction. Active noise reduction (also referred to as “active noise control”, “noise cancellation” or other similar term) refers to reducing an unwanted first sound by the addition of a second sound specifically designed to cancel the first. Active noise reduction requires the creation of the second sound and therefore requires some device or hardware for producing the second sound. Typically, such a device or hardware includes both a microphone to receive the first sound and a speaker to produce the second sound. Preferably, the earplug of the present application does not have a device or hardware for active noise reduction. In preferred embodiments, the earplug of the present application is devoid of electronic components. Such electronic components may include a microphone, a speaker, a battery, a controller or control board, or other similar electronic components.

In some embodiments, the earplug can be connected to another earplug as described above by a connecting cord to form a pair of earplugs. The pair of earplugs together with the connecting cord can form a hearing protection device. In general, the connecting cord can be any suitable connecting cord. That is, the connecting cord can include or be formed of any suitable material for forming a cord, such as metal links to form a chain, fabric or rope, or a flexible plastic or elastomer as described above. A fabric or rope can be formed of or include any suitable material, such as cotton, flax, hemp, jute, sisal, kapok, alpaca hair, camel hair, llama hair, mohair, silk, synthetic fibers such as aramid, lastrile, melamine, novoloid, nylon, polyester, and acrylic, natural polymeric fibers such as rayon, and the like. In some embodiments, the cord can be connected to the stopper portion of an earplug.

The examples below are intended to further illustrate protocols for constructing the earplug of the present disclosure and are not intended to limit the scope of the claims.

Where a numerical limit or range is stated herein, the endpoints are included. Also, all values and subranges within a numerical limit or range are specifically included as if explicitly written out.

Numerous modifications and variations of the present invention are possible in light of the above teachings. It is therefore to be understood that, within the scope of the appended claims, the invention may be practiced otherwise than as specifically described herein.

### EXAMPLES

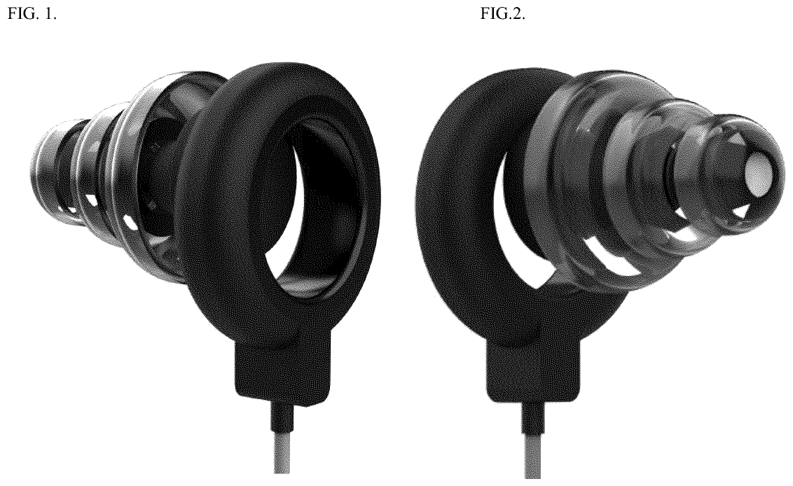

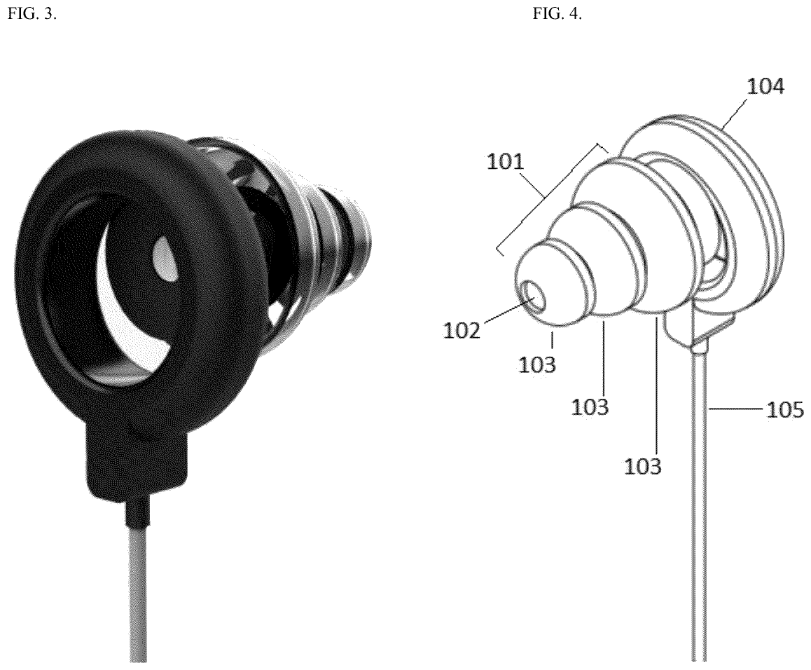

FIG. 1 shows a perspective rendering of a rear three-quarters view of an earplug according to the present disclosure. FIG. 2 shows a perspective rendering of front three-quarters view of an earplug according to the present disclosure. FIG. 3 shows an alternate perspective rendering of a rear three-quarters view of an earplug according to the present disclosure.

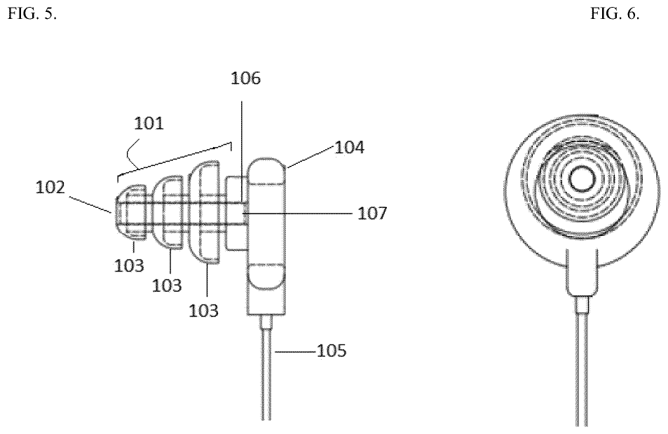

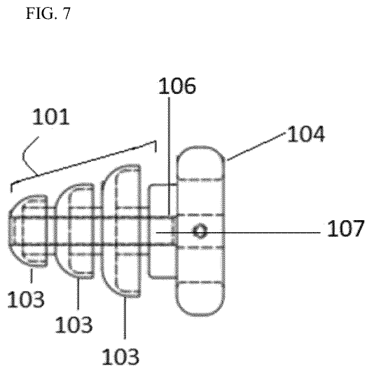

FIGS. 4-7 show schematic drawings of the earplug according to an exemplary embodiment. FIG. 4 shows a front three-quarters view of an earplug according to the present disclosure. FIG. 5 shows a side-view of an earplug according to the present disclosure. FIG. 6 shows a front view of an earplug according to the present disclosure. FIG. 7 shows a top-down view of an earplug according to the present disclosure. The figures show the tapered body portion 101. Centered in the tapered body portion 101 is the bamboo attenuator 102. The tapered body portion 101 is shown having a cylindrical shape with a bamboo attenuator 102 also having a cylindrical shape with a circular cross-section. Disposed around the tapered body portion 101 are three contact structures 103 of increasing size moving toward the stopper portion 104. The connecting cord 105 is shown connected to the bottom of the stopper portion 104. In the exemplary embodiment depicted in FIG. 4, the bottom of the stopper portion is oriented toward an earlobe of a user wearing the earplug. The exemplary embodiment depicted in FIGS. 5 and 7 also show the central shaft 106 and hollow sheath 107 in which the bamboo attenuator 102 is disposed.

Attenuation characteristics of exemplary earplugs depicted in the figures is summarized in Table 1 below.

TABLE 1
Exemplary Earplug Attenuation Characteristics.

| Bamboo rod size | Sound Level Permitted | Sound Level Blocked |
|---|---|---|
| 2 mm | 0-68 db | Above 68 db |
| 3 mm | 0-75 db | Above 75 db |
| 4 mm | 0-89 db | Above 89 db |

Attenuation characteristics of exemplary earplugs using different species of bamboo is summarized in Table 2 below.

| | Maximum | Estimated Lifetime |
|---|---|---|
| | Decibel | (under optimal |
| Bamboo Type | Level | conditions) |
| Acidosasa | 78.2 db | 4 days |
| Dracaena sandriana | 75 db | 3 weeks |
| Dendrocalamus hamiltonii | 90.5 db | 4 weeks |

1. An earplug, comprising:
a tapered body portion, comprising:
a central shaft comprising a hollow sheath and a bamboo attenuator disposed within the hollow sheath, and
a plurality of contact structures arranged about an exterior of the central shaft, the contact structures being flexible and having a successively smaller size moving from a lateral end of the tapered body portion to a medial end of the tapered body portion; and
a stopper portion attached to the lateral end of the tapered body portion, wherein
the earplug is configured to be placed in an ear such that the medial end of the tapered body portion is securely disposed within an ear canal by contact between the contact structures and an interior surface of the ear canal and
the stopper portion is rigid and securely disposed in an exterior portion of the ear,
wherein the bamboo attenuator is a rod which extends throughout an entirety of a length of the central shaft.

2. The earplug of claim 1, wherein the earplug comprises at least three contact structures.

3. The earplug of claim 1, wherein the contact structures are formed from an elastomer.

4. The earplug of claim 3, wherein the elastomer is a silicone elastomer.

5. The earplug of claim 1, wherein the stopper portion is formed from a rigid material.

6. The earplug of claim 5, wherein the rigid material is a rigid rubber.

7. The earplug of claim 1, wherein each of the contact structures is disposed about an entirety of a circumference of the central shaft.

8. The earplug of claim 7, wherein the contact structures have an elliptical shape about the exterior of the central shaft.

9. The earplug of claim 7, wherein the contact structures have a rounded trough cross-sectional shape having a rounded trough bottom oriented toward the medial end of the tapered body portion and an open trough top oriented toward the lateral end of the tapered body portion.

10. The earplug of claim 1, wherein the bamboo attenuator is a cylindrical rod.

11. The earplug of claim 10, wherein the bamboo attenuator has a diameter of 1.5 to 5.0 mm.

12. The earplug of claim 1, wherein the earplug is devoid of electrical components.

13. The earplug of claim 1, wherein the tapered body portion has a length of 15.0 to 20.0 mm.

14. The earplug of claim 1, wherein the earplug has a total length of 18.0 to 28.0 mm.

15. The earplug of claim 8, wherein a lateralmost contact structure has a major axis of 11.0 to 17.0 mm.

16. The earplug of claim 8, wherein a medialmost contact structure has a major axis of 5.0 to 10.0 mm.

17. The earplug of claim 1, having a threshold attenuation level of 60 to 100 db.

18. A hearing protection device, comprising:
a first earplug;
a second earplug; and
a connecting cord attached to the stopper portion of each of the first earplug and the second earplug, wherein
the first earplug and second earplug are each an earplug of claim 1.
